(19) United States
(12) Patent Application Publication
(10) Pub. No.: US 2013/0095492 A1
(43) Pub. Date: Apr. 18, 2013

(54) METHOD OF DETECTING TAU PROTEIN AND TAU FRAGMENTS IN SERUM

(71) Applicants: John F. DeBernardis, Lake Zurich, IL (US); Raymond P. Zinkowski, Lindenhurst, IL (US)

(72) Inventors: John F. DeBernardis, Lake Zurich, IL (US); Raymond P. Zinkowski, Lindenhurst, IL (US)

(73) Assignee: ALBERT EINSTEIN COLLEGE OF MEDICINE OF YESHIVA UNIVERSITY, Bronx, NY (US)

(21) Appl. No.: 13/581,485

(22) PCT Filed: Mar. 4, 2011
(86) PCT No.: PCT/US2011/000419

§ 371 Date: Dec. 4, 2012

Related U.S. Application Data
(60) Provisional application No. 61/311,140, filed on Mar. 5, 2010.

(51) Int. Cl. G01N 33/68

(52) U.S. Cl. CPC G01N 33/6896
USPC 435/6.12; 436/501; 435/7.1; 435/7.92

(57) ABSTRACT

The present invention provides quantitative methods for the detection of tau protein and/or tau fragments. More specifically, the present invention provides quantitative methods for diagnosing a tauopathy or ruling out a tauopathy as the cause of disease, particularly the diagnosis and ruling of Alzheimer's disease. The present invention further provides a method for diagnosing a tauopathy by computing the ratio of two detected tau proteins or tau fragments.

### CROSS-REFERENCE TO RELATED APPLICATION

This application claims the benefit of U.S. Provisional Application No. 61/311,140, filed Mar. 5, 2010, the contents of which is hereby incorporated by reference into the subject application.

### TECHNICAL FIELD

The present invention is directed to the detection of tau fragments and tau proteins found in biological samples to detect tauopathies. Specifically, the present invention provides a method for the detection and/or diagnosis of tauopathies. More specifically, the invention relates to computing the ratio of two detected tau proteins or tau fragments to better diagnosis tauopathies including Alzheimer's disease.

### BACKGROUND ART

Several tauopathies have been associated with the same pathophysiological mechanism, the involvement of the structural protein tau. Tau protein is a microtubule-associated protein located primarily in the neurons of the central nervous system. The tau gene is located on the long arm of chromosome 17 and alternative mRNA splicing results in six brain tau proteins that are normally expressed in the adult human central nervous system.

In non-diseased brains, tau protein serves to stabilize the microtubules through interaction with tubulin. In diseased states, tau protein undergoes abnormal phosphorylation, enzymatic cleavage, and conformational change; all events which contribute to the insoluble accumulation of misfolded tau. The abnormal regulation of enzymatic cleavage generates truncated tau subproducts (tau fragments) which in turn may be toxic to neurons per se and capable of polymerization at a faster rate. Specifically, the fragmented state of tau may influence the normal functions and characteristics of the tau protein including its ability to associate with and support microtubules, and abnormal events such as self assembly into filaments and ability to induce apoptosis.

The tau protein has been shown to play a significant role in Alzheimer's disease (AD). A definitive feature of AD is an increased presence of neurofibrillary tangles within the affected cortical regions of the brain. Research has demonstrated that the neurofibrillary tangles constitute paired helical filaments and tau proteins. Corresponding to the increased presence of tangles are significantly higher levels of tau in the CSF of AD patients compared to tau levels in the CSF of non-demented controls.

Elevated tau protein levels are also present where neuronal death or damage has occurred. For example tau CSF levels have been shown to be elevated in stroke, Creutzfeldt-Jakob disease, multiple sclerosis, vascular dementia, and dementia with Lewy Bodies.

The increased levels of total tau protein and the presence of cleaved tau have both been utilized as diagnostic markers of Alzheimer's disease. A number of diagnostic methods using tau have been demonstrated. For example, levels of phosphorylated tau in the CSF have been measured and the ratio of total tau to an Aβ 1-42 protein have been measured. Standing alone, increased levels of tau protein and cleaved tau cannot definitively diagnosis AD due to elevated levels of tau protein and cleaved tau in various other diseases.

### SUMMARY OF THE INVENTION

The present invention provides quantitative methods of determining the amount of tau protein and tau fragments in a biological sample. The invention also provides methods for detecting a tauopathy or ruling out a tauopathy as a cause of disease. Specifically, the ratio of at least two tau fragments can be used as a diagnostic tool for tauopathies, particularly Alzheimer's disease.

In one aspect, the invention provides quantitative methods of determining the amount of at least one tau protein or tau fragment in a biological sample. The methods involve contacting the sample with a first antibody and a second antibody to form a complex-where both the first antibody and the second antibody bind specifically to tau polypeptide and detecting the presence or amount of the complex where the presence or amount of the complex is indicative of the presence or amount of tau polypeptide.

In some aspects, the first antibody can be specific to any of tau epitopes 180-210, 200-250, 102-150 and the second antibody can be specific to tau epitope 150-170. In other aspects, the first antibody is specific to any of tau epitopes 192-204, 210-230, 102-150 and the second antibody is specific to tau epitope 159-163.

In one aspect of the invention, the biological sample is whole blood, serum or plasma. In another aspect of the invention, the biological sample is cerebral spinal fluid.

In certain aspects of the invention, the first antibody has specificity to Tau epitopes that correspond to SEQ ID Nos. 1, 2, 5, 6 or 7 and the second antibody has specificity to Tau epitopes that correspond to SEQ ID Nos. 3 or 4.

The invention also provides methods of detecting a tauopathy. The methods involve contacting the sample with a first antibody and a second antibody to form a complex where both the first antibody and the second antibody bind specifically to tau polypeptide and detecting the presence or amount of the complex where the presence or amount of the complex is indicative of the presence or amount of tau polypeptide and determining the ratio of at least two detected tau fragments or tau proteins to diagnose a tauopathy or to rule out a tauopathy as the cause of disease.

The invention provides methods of detecting a tauopathy comprising the steps of determining the amount of a tau protein or tau fragment in a sample and determining the ratio of at least two tau fragments or tau proteins.

The invention also provides methods of detecting a tauopathy by determining the amount of a tau protein or a tau fragment in a biological sample comprising contacting the sample with a first antibody and a second antibody to form a complex where both the first antibody and the second antibody bind specifically to tau polypeptide and detecting the presence or amount of the complex where the presence or amount of the complex is indicative of the presence or amount of tau polypeptide and determining the ratio of at least two detected tau fragments or tau proteins.

Specific preferred embodiments of the invention will become evident from the following more detailed description of certain preferred embodiments and the claims.

### DETAILED DESCRIPTION OF THE DRAWINGS

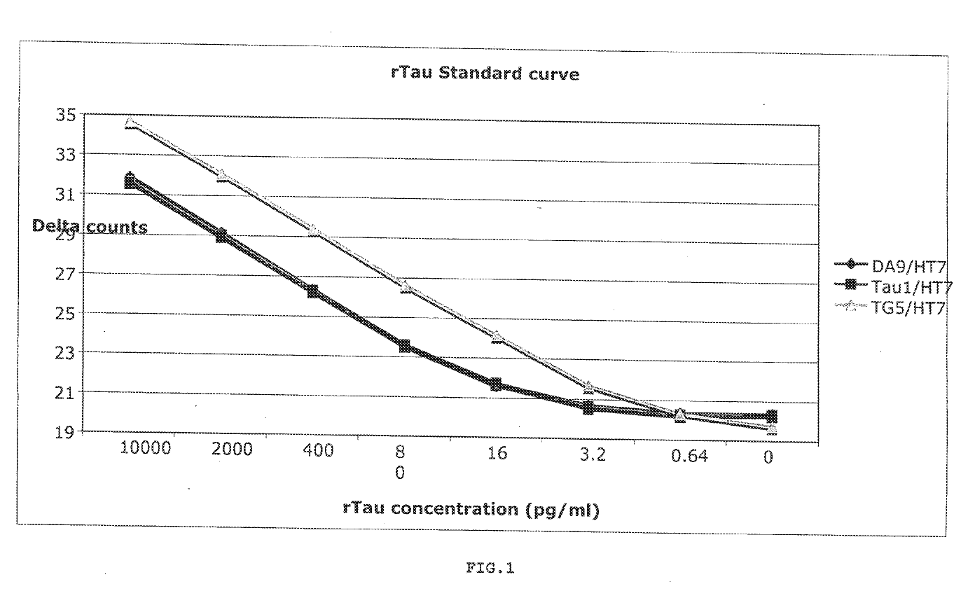

FIG. 1: The standard curve for each of the three sandwich assays (capture antibodies Taul, DA9 and TG5, all using HT7 as a detection antibody) using full-length recombinant tau diluted in synthetic serum (Biseko®). The rTau concentration is plotted in 9g/ml versus delta counts.

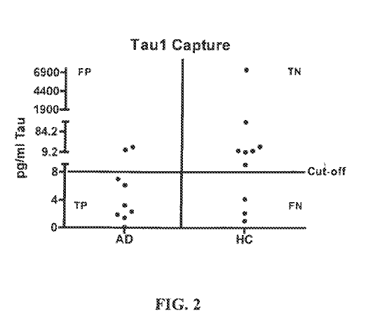

FIG. 2: A scattergram showing individual data points for AD and control sera from an assay using TG5 capture antibody and HT7 detection antibody. The cutoff value determined from the ROC curve analysis was 0.34 pg/ml using the maximum sum of sensitivity and specificity. The sensitivity of this assay was 83.3% with a specificity of 42.9%. Samples with undetectable tau levels were set to 0 pg/ml.

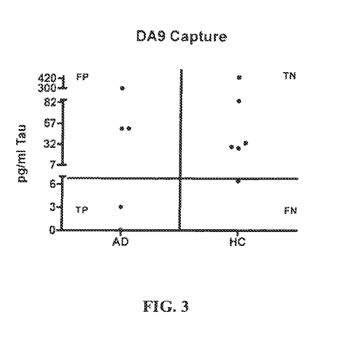

FIG. 3: A scattergram showing individual data points for AD and control sera from an assay using Taul capture antibody and HT7 detection antibody. The cutoff value determined from the ROC curve analysis was 9.03 pg/ml using the maximum sum of sensitivity and specificity. The sensitivity of this assay was 88.9% with a specificity of 50%. Samples with undetectable tau levels were set to 0 pg/ml.

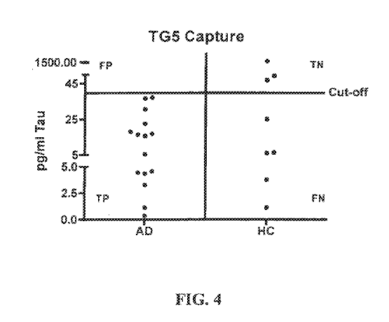

FIG. 4: A scattergram showing individual data points for AD and control sera from an assay using DA9 capture antibody and HT7 detection antibody. The cutoff value determined from the ROC curve analysis was 6.48 pg/ml using the maximum sum of sensitivity and specificity. The sensitivity of this assay was 83.3% with a specificity of 42.9%. Samples with undetectable tau levels were set to 0 pg/ml.

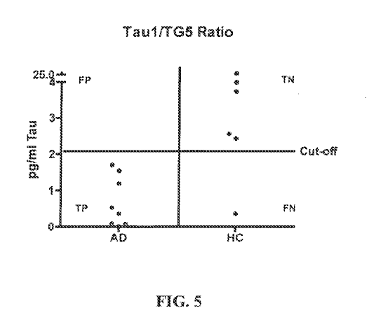

FIG. 5: Scattergrams showing a subset of data points from the individual assays and the calculated ratio of a TG5 assay relative to a Taul assay. TG5 is an assay that includes a TG5 capture antibody and HT7 detection antibody with a subset of data points from the individual assay of FIG. 2 shown in 5A. The sensitivity of this assay was 87.5% with a specificity of 50% using a cutoff value of 25.01 pg/ml. Taul is an assay that includes a Tau 1 capture antibody and HT7 detection antibody with a subset of data points from the individual assay of FIG. 3 shown in 5B. The sensitivity of this assay was 75% with a specificity of 100% using a cutoff value of 9.03 pg/ml. The calculated ratio of a TG5 assay relative to a Taul assay is shown in 5C. The sensitivity of this assay was 100% with a specificity of 83.3%. The cutoff value determined from the ROC curve analysis was a ratio of 0.41. In all cases, the cutoff values were determined from ROC curve analysis when the sum of the sensitivity and specificity were maximized.

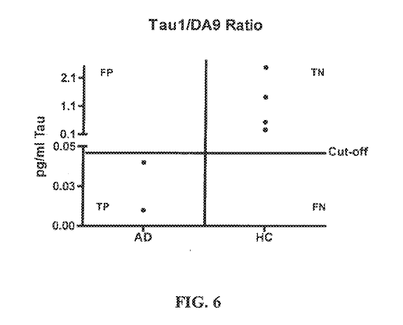

FIG. 6: Scattergrams showing a subset of data points from the individual assays and the calculated ratio of a TG5 assay relative to a DA9 assay. TG5 is an assay that includes a TG5 capture antibody and HT7 detection antibody with a subset of data points from the individual assay of FIG. 2 shown in 6A. The sensitivity of this assay was 100% with a specificity of 50% using a cutoff value of 6.31 pg/ml. DA9 is an assay that includes a DA9 capture antibody and HT7 detection antibody with a subset of data points from the individual assay of FIG. 4 shown in 6B. The sensitivity of this assay was 40% with a specificity of 83.3% using a cutoff value of 6.48 pg/ml.. The calculated ratio of a TG5 assay relative to a DA9 assay is shown in 6C. The sensitivity of this assay was 80% with a specificity of 66.7%.The cutoff value determined from the ROC curve analysis was a ratio of 0.58. In all cases, the cutoff values were determined from ROC curve analysis when the sum of the sensitivity and specificity were maximized.

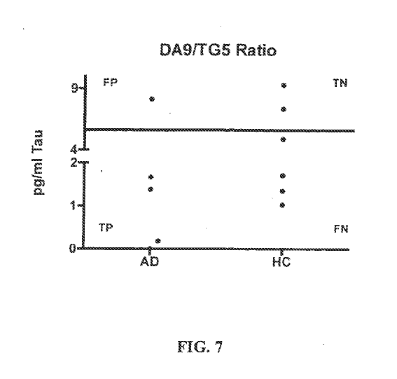

FIG. 7: Scattergrams showing a subset of data points from the individual assays and the calculated ratio of a DA9 assay relative to a Taul assay. DA9 is an assay that includes a DA9 capture antibody and HT7 detection antibody with a subset of data points from the individual assay of FIG. 4 shown in 7B. The sensitivity of this assay was 50% with a specificity of 100% using a cutoff value of 6.48 pg/ml. Tau 1 is an assay that includes a Taul capture antibody and HT7 detection antibody with a subset of data points from the individual assay of FIG. 3 shown in 7A. The sensitivity of this assay was 100% with a specificity of 100% using a cutoff value of 9.03 pg/ml. The calculated ratio of a DA9 assay relative to a Taul assay is shown in 7C. The sensitivity of this assay was 100% with a specificity of 100%.The cutoff value determined from the ROC curve analysis was a ratio of 3.74. In all cases, the cutoff values were determined from ROC curve analysis when the sum of the sensitivity and specificity were maximized.

### DETAILED DESCRIPTION OF THE INVENTION

In one aspect, the present invention provides quantitative methods of measuring the amount of tau fragments or tau protein in a sample. The methods involve contacting the sample with a first antibody and a second antibody to form a complex where both the first antibody and the second antibody bind specifically to a tau polypeptide, and detecting the presence or amount of the complex where the presence or amount of the complex is indicative of the presence or amount of tau polypeptide in the sample. As used herein, the term tau protein refers to any of the six non-cleaved isoforms of the protein that have a molecular weight in the range of 48 to 68 kDa. Full-length human tau protein contains 441 amino acids. As used herein the term tau fragment refers to a protein with a reduced molecular weight compared to tau protein and can be comprised of the interior portion of the tau protein sequence that includes Lys¹⁸⁰-Ser²¹⁰, Gly¹⁹²-G1y²⁰⁴, Pro²⁰⁰-Met²⁵⁰, Ser²¹⁰-Arg²³⁰, Lys¹⁵⁰-Pro¹⁷⁰, Pro¹⁵⁹-Lys¹⁶³, Thr¹⁰²-Lys¹⁵⁰. Illustrative examples of tau fragments are SEQ ID NOs. 1, 2, 3, 4, 5, 6, and 7.

Tau Peptides

The quantitative methods of the invention can be adopted for measuring any tau peptide or tau fragment and are particularly suitable for measuring tau fragments that contain epitopes on the tau protein in the regions of 102-170, 180-250. The term epitope refers to that portion of a molecule that is specifically recognized by an antibody and the site on the molecule in which that antibody binds. As used herein specifically recognized is the substantial recognition of an antibody for an epitope where the antibody is complementary to an area on the epitope. Methods of the invention can be adopted for measuring the amount of one tau fragment, total amount of a plurality of tau fragments or a ratio of a plurality of tau fragments in a sample.

Sample Collection and Preparation

The quantitative methods of the invention can be adopted for use with any sample where tau protein or tau fragments are present. The methods are particularly suitable for measuring tau fragments in biological samples. Examples of suitable samples include biological fluids such as whole blood, serum, plasma, urine, lymph, and cerebrospinal fluid; blood components such as plasma, serum, blood cells, and platelets; and cultures of human or animal cell lines or primary cells. Methods of the invention are particularly suitable for measuring tau fragments in a sample of blood, such as whole blood, plasma, serum, or a sample containing any blood component in any amount.

Biological samples of whole blood can be collected using any suitable method known in the art. For example, a sample of blood can be removed from the subject by venesection. A blood sample may or may not be placed in contact with an anticoagulant depending on whether separation of blood components is required. Any suitable anticoagulants may be used in blood collection. Illustrative examples of suitable anticoagulants include ethylenediaminetetraacetate (EDTA), heparin and citrate. Plasma and cellular components may be separated from the whole blood by any method know in the art such as centrifugation and filtration.

Contacting the Sample with Abs

Many methods are known for generating and/or identifying antibodies to a given target peptide. In general, to generate antibodies, an isolated peptide or DNA encoding the peptide is used as an immunogen and is administered to a mammalian organism, such as a rat, rabbit or mouse. The full-length protein, an antigenic protein fragment or a fusion protein can be used as antigens.

In one aspect of the invention, antibodies are prepared from regions or discrete fragments of the tau proteins. Antibodies can be prepared from any of the protein or protein fragments as described herein.

An antigenic fragment will typically comprise at least 8 contiguous amino acid residues. The antigenic peptide can comprise, however, at least 10, 12, 14, 16 or more amino acid sequences. Such fragments can be selected on a physical property.

The first and second antibodies of the invention can be produced using procedures well-known in the art. In one aspect of the invention, a monoclonal antibody to a peptide of tau may be prepared using hybridoma cell lines including B-cell hybridomas and EBV hybridoma cells. In another aspect, a polyclonal antibody to a peptide of tau may be prepared using a host animal which may be immunized by injection with a particular tau protein or tau fragment. The host animal may include but is not limited to a rabbit, mouse, goat, chicken, or rat. To increase the immunological response in the host animal, various adjuvants may be used such as Freund's, mineral gels, surface active substances, and potentially useful human adjuvants.

In one aspect of the invention, the first antibody is specific to the tau epitope 180-210 and the second antibody is specific to the tau epitope 150-170. In another aspect of the invention, the first antibody is specific to the tau epitope 192-204 and the second antibody is specific to the tau epitope 159-163. In another aspect of the invention, the first antibody is specific to the tau epitope 200-250 and the second antibody is specific to the tau epitope 150-170. In yet another aspect of the invention, the first antibody is specific to the tau epitope 210-230 and the second antibody is specific to the tau epitope 159-163. In yet another aspect of the invention, the first antibody is specific to the tau epitope 102-150 and the second antibody is specific to tau epitope 150-170 or 159-163

Details of the characterization of antibodies have been described for TG5 (Carmel et al., 1996, J. Biol. Chem. 271: 32789-32795) and Taul (Szendrei et al., J. Neurosci. Res. 34: 243-249). Taul is commercially available from Millipore and HT7 is commercially available from Thermo Fisher Scientific.

The complexes formed in the quantitative methods of the invention comprise the first antibody, a tau fragment and a second antibody. The complex is created when the first antibody binds to a tau fragment and the second antibody binds to the same tau fragment at an epitope distinct from the binding site of the first antibody. The first antibody and second antibody do not compete for binding on the same epitope. The complex will form under conditions that respect the immunological properties of the antibodies and the tau fragments. Examples of factors that can be varied to allow complex formation include temperature, time of incubation, concentrations of the first and second antibody as well as other assay conditions.

Detecting the Complex

In one aspect of the invention, detecting the presence or amount of the complex includes real-time PCR amplification of an amplifiable DNA conjugated to the second antibody. The second antibody can have specificity for tau epitopes 150-170 and 159-163. The suitability and use of real-time PCR amplification of an amplifiable DNA conjugated to an antibody are discussed in M. Adler, et al., “A real-time immuno-PCR assay for routine ultrasensitive quantification of proteins” Biochem Biophys Res Commun. 2003:308(2):240-250; Niemeyer, et al., “Detecting antigens by quantitative immuno-PCR” Nat. Protoc. 2007, 2(8):1918-1930; M. Adler, et al., “Sensitivity by Combination: Immuno-PCR and Related Technologies” The Analyst. 2008, 133:702-18 which are incorporated by reference herein in their entirety.

In another aspect of the invention, detecting the presence or amount of the complex includes enzyme amplification. Enzyme amplification can occur when the second antibody is linked to an enzyme. Detection will occur when an appropriate substrate is introduced wherein the enzyme converts the substrate to a detectable product. Illustrative examples of suitable substrates include fluorescent substrates, chemiluminescent substrates and chromogenic substrates. Suitable enzymes include but are not limited to horseradish peroxidase, alkaline phosphatase, β-galactosidase, or acetylcholinesterase.

In another aspect of the invention, detecting the presence or amount of the complex includes readout by fluorescence. For example, readout by fluorescence can occur when a non-enzyme light emitting label is bound to the second antibody. The non-enzyme light emitting label can be any fluorescent label known within the art. A non-enzyme light emitting label as used herein refers to any label that does not include an enzyme and is capable of producing a signal by itself. Illustrative examples of a non-enzyme light emitting label include fluorescein, and rhodamine. A fluorescently labeled second antibody can be detected when the fluorescent label is exposed to a light of the proper wavelengh and can be quantified using known methods in the art including a fluorometer. Readout by fluorsecence can also include enzyme amplification of a fluorogenic substrate. The relative fluorescence units (emitted photons of light) that are detected are typically proportional to the amount of analyte being measured. Suitable enzymes for enzyme amplification of a fluorogenic substrate include but are not limited to alkaline phosphatase, β-galactosidase or peroxidase. A fluorogenic substrate may be chosen for its quantitative emission of light following excitation. Suitable substrates include but are not limited to 4-methylumbelliferyl phosphate, 4-methylumbelliferyl galactoside, hydroxyphenylacetic acid, 3-p-hydroxyphenylproprionic acid.

In another aspect of the invention, detecting the presence or amount of the complex includes readout by luminescence detection. Readout by luminescence may include the presence of a luminescent-tagged second antibody that can be detected by the presence of luminescence that occurs during the course of a chemical reaction. Examples of luminescent labeling compounds include but are not limited to luminol, isoluminol, acridinium ester, imidazole, acridinium salt and oxalate ester. Luminescence detection can also include enzyme amplification wherein an enzyme converts a substrate to a reaction product that emits photons of light instead of developing a visible color. Luminescence can be bioluminescence, chemiluminescence, photoluminescence each which differ in the way the excited state is reached. For each type of luminescence a suitable enzyme includes alkaline phosphatase, β-galactosidase or peroxidase. Suitable substrates include luminol, polyphenols and acridine esters, luciferin. pyrogallol, purpurogallin, gallic acid, umbelliferone, 3-(2′-spiroadamantane)-4-methyl-4-(3′-phosphoryloxyphenyl-1, 2-dioxetane, disodium salt and 3-(2′-spiroadamantane)-4-methoxy-4-(3′-β-D-gal actopyranosyloxyphenyl-1,2-dioxetane).

In yet another aspect of the invention, detecting the presence or amount of the complex includes colorimetric detection. Colorimetric detection results in a colored reaction product that absorbs light in the visible range. For colorimetric detection the rate of color development is proportional, over a certain range, to the amount of enzyme conjugate present. Suitable enzymes for colorimetric detection include but are not limited to alkaline phosphatase, B-galactosidase or peroxidase. Suitable enzymes include but are not limited to 5-bromo-4-chloro-3-indolyl-phosphate/nitroblue tetrazolium, p nitrophenylphosphate, 3,3′,5,5′ tetramethylbenzidine, 3,3′,4,4′ diaminobenzidine, 4-chloro-1-naphthol, TMB (dual function substrate), 2,2′-azino-di [3-ethylbenzthiazoline and o-phenylenediamine.

Raw data generated through the various detection methods can be converted to pg/ml by using a standard curve. Such a standard curve may be prepared by spiking human serum with full length recombinant tau varying in concentrations up to lOng/ml.

Immobilization of Antibody on a Solid Substrate

In some aspects of the invention the first antibody is immobilized on a solid substrate and the second antibody comprises a label, where the presence or amount of the complex is measured by the amount of signal from the label bound to the second antibody. It would be clear to one skilled in the art that either the first or second antibody could be immobilized on the solid substrate.

A suitable example of a solid substrate includes but is not limited to, glass and polymers. Examples of polymers include but are not limited to cellulose, polyacrylamide, nylon, polystyrene, polyvinylchloride or polypropylene. The quantitative methods of the invention can be adopted for use with solid substrates in the form of tubes, beads, discs, microplates or any surface that is a suitable surface for conducting an immunoassay.

In one aspect where the first antibody is immobilized on a solid substrate, the first antibody (also called “capture antibody”) captures tau fragments or tau protein from the sample. The capture antibody is specific to a target tau fragment or tau protein. The captured tau fragment is detected using a second antibody that is specific for a tau fragment or tau protein (also called a detection antibody). The first antibody and the second antibody do not compete for binding to the tau protein or tau fragment. The second antibody can also function as a capture antibody and the first antibody can function as a detection antibody.

As used herein, an antibody “comprising a label” refers to an antibody that is directly or indirectly labeled. A “label” is any molecule that is bound to another molecule that allows detection of the labeled molecule. Examples of suitable labels include, but are not limited to, particulate metal and metal derivatives, radioisotopes, enzyme substrates, enzymes, chromogenic substrates and chromophores, fluorescent and chemiluminescent molecules and phosphors. A directly labeled antibody is an antibody that comprises of a label that emits a signal by itself and such signal may be detected by electromagnetic radiation or visual examination. Illustrative examples of labels that emit a signal by itself include but are not limited to fluorescent labels and radioactive labels.

Alternatively, a label may require other components to produce a signal, such as substrates or enzymes. Suitable examples of an enzyme include but are not limited to, horseradish peroxidase, glucose oxidase, β-galactosidase and alkaline phosphatase. Suitable substrates include those substrates that produce a detectable color change upon hydrolysis by the corresponding enzyme. In one aspect of the invention, the enzyme-labeled second antibody is added to the first antibody-tau fragment complex and allowed to bind to the complex. The excess reagent will be washed away and an appropriate substrate is added to the complex comprising of the first antibody, the tau fragment and the second antibody. The appropriate substrate will react with the enzyme and produce a qualitative visual signal that is capable of being quantified by methods such as spectrophotometry.

In another aspect, the method further comprises the step of removing any unbound material from the solid substrate. If the second antibody is bound by a label that emits a signal by itself the solid substrate is washed after the second antibody is added to the complex and the unbound second antibody is removed.

Diagnosing a Tauopathy

In one aspect of the invention, a method for determining the amount or presence of at least one tau protein or tau fragment in a biological sample comprises the steps contacting the sample with a first antibody and a second antibody to form a complex where both the first antibody and the second antibody bind specifically to tau polypeptide and detecting the presence or amount of the complex, where the presence or amount of the complex is indicative of the presence or amount of tau polypeptide further comprising the step of determining the ratio of at least two detected tau fragments or tau proteins to diagnose a tauopathy or to rule out a tauopathy. In another aspect of the invention, a method for determining the amount or presence of at least one tau protein or tau fragment in a biological sample comprises the steps contacting the sample with a first antibody and a second antibody to form a complex where both the first antibody and the second antibody bind specifically to tau polypeptide and detecting the presence or amount of the complex, where the presence or amount of the complex is indicative of the presence or amount of tau polypeptide and determining the ratio of at least two detected tau fragments or tau proteins to rule out Alzheimer's Disease in a patient.

A tauopathy is any form of dementia that is associated with a tau pathology. For example, Alzheimer's disease, Pick's disease, sporadic Frontotemporal dementia and Frontotemporal dementia with Parkinsonism linked to chromosome 17 are common forms of tauopathy.

A tauopathy can be ruled out by detecting levels of tau fragments or tau protein and using a cut off value to separate normal individuals from those with AD that is calculated based on sensitivity and specificity. In some aspects of the invention, ruling out of a tauopathy can include ruling out Alzheimer's disease in a patient. The patient can be a healthy individual or an individual with symptoms of a tauopathy.

In certain aspects of the invention, the ratio is a detected tau fragment using a first antibody specific to tau epitope 200-250 and a second antibody specific to 150-170 relative to a second detected tau fragment using a first antibody specific to tau epitope 180-210 and a second antibody specific to 150-170. In other aspects of the invention, the ratio is a detected tau fragment using a first antibody specific to tau epitope 210-230 and a second antibody specific 159-163 relative to a second detected tau fragment using a first antibody specific to tau epitope 192-204 and a second antibody specific to 159-163. In other aspects of the invention, the ratio is a detected tau fragment using a first antibody specific to tau epitope 102-150 and a second antibody specific 150-170 relative to a second detected tau fragment using a first antibody specific to tau epitope 180-210 and a second antibody specific to 150-170. In other aspects of the invention, the ratio is a detected tau fragment using a first antibody specific to tau epitope 102-150 and a second antibody specific 159-163 relative to a second detected tau fragment using a first antibody specific to tau epitope 192-204 and a second antibody specific to 159-163. In yet other aspects of the invention, the ratio is a detected tau fragment using a first antibody specific to tau epitope 200-250 and a second antibody specific 150-170 relative to a second detected tau fragment using a first antibody specific to tau epitope 102-150 and a second antibody specific to 150-170. In yet another aspect of the invention, the ratio is a detected tau fragment using a first antibody specific to tau epitope 210-230 and a second antibody specific 159-163 relative to a second detected tau fragment using a first antibody specific to tau epitope 102-150 and a second antibody specific to 159-163.

Diagnosis or ruling out of a tauopathy can include any diagnostic tool that can be used to separate data points of healthy individuals from data points of diseased patients. Such detection tools can be based on optimizing either specificity or sensitivity or both. In one aspect of the invention, detection or the ruling out of a tauopathy includes use of Receiver Operating Characteristic (ROC) curve analysis. ROC curve analysis is a graphical plot of sensitivity vs. (1-specificity). The cut-off value for the assay can be derived from the ROC curve analysis at a point where the sum of specificity and sensitivity is maximized. In one aspect of the invention, the data for the ratio of two assays can be plotted in a scattergram to demonstrate the relationship between the concentration of detected tau and the diagnosis of AD or the ruling out of AD (FIGS. 2-7).

In another aspect of the invention, a method of detecting a tauopathy comprises the steps of determining the amount of a tau protein or tau fragment in a sample and determining the ratio of at least two tau fragments or tau proteins. Determining the amount of tau protein or tau fragment can be by any method known in the art, including but not limited to use of antibodies or by functional assays. Illustrative examples of determining the amount of a tau protein or fragment in a sample include but are not limited to western blots, immunodiffusion methods and magnetic immunoassays.

### EXAMPLES

### Example 1

### Description of Samples

Clinical blood samples were obtained from The Dementia Research Section and Memory Clinic, Alzheimer Memorial Center and Geriatric Psychiatry Branch, Department of Psychiatry, Ludwig-Maximilian University, Munich Germany. The samples were taken from patients that were clinically diagnosed as AD or normal controls.

### 1. Human TAU His-Tag Immuno-PCR Assay

A monoclonal anti-Tau antibody is immobilized onto a microplate and the plate is blocked for unspecific binding. Standards and samples are mixed with a solution of an antibody-DNA detection conjugate specific to tau and pipetted into the microplate coated with the capture antibodies.

To quantify levels of tau in each sample real-time PCR is performed using a dual-labeled probe. The probe contains a fluorescence dye (FAM) and a quencher. While the DNA-polymerase elongates the PCR-primer during the synthesis of the novel complementary DNA strand, the probe becomes sterically altered, which induces fluorescence for each amplified DNA strand. Signal readout of the real-time IPCR is done according to cycler manual. A Stratagene MX3000/3005 real-time PCR cycler is used. The readout can be in delta counts (real time PCR), light units (luminescence and fluorescence), or optical density (OD) for colorimetric assays etc.

In the sections below, a detailed description is provided for the methods used for the Immuno-PCR assay.

### Plate Preparation

The capture antibody is diluted (Taul or TG5 or DA9) in coating buffer. A working concentration of 5 μg/ml is used for Taul and DA9 and 10 μg/ml is used for TG5. 30 μl/well of the capture antibody dilution is added to the TopYield™ modules and the modules are sealed with cling film and incubated for 12-48 h at 4° C. The minimum incubation time is 12 hours, however the coating step can be carried out as long as 48 hours with no detectable change in the assay. Directly before the samples and standards are added to the wells, the wells are washed three times for one minute with 240 μl/well Wash-Buffer A at room temperature and orbital shaking. Next, 240 μl/well of the Chimera Direct Block is added for one minute at room temperature with orbital shaking.

Preparation of Dilution Series and Coupling with Imperacer® Conjugate:

For calibration purposes, standard curves are run for each of the three sandwich assays (capture antibodies Taul, DA9, and TG5, all using HT7 as the detection antibody). Calibration samples are prepared using full-length recombinant tau diluted into a synthetic blood serum (Biseko®). Samples can include concentrations of Tau up to 10 ng/ml. In one example, a data point that includes no antigen is included in the standard curve.

A 1:100 dilution of the Imperacer® conjugate CHI-TAU HT7 in SDB₂₁₀₀ is mixed by addition of 6 μl conjugate to 594 μl SDB_{2100. 60} μl of the diluted conjugate is then added to 15 μl of each sample to be analyzed to achieve a 1+4 assay mixture and the samples are thoroughly mixed. Next, the capture-antibody coated and blocked modules are washed twice for 30 seconds and twice for 4 minutes with 240 μl/well Wash-Buffer B at room temperature and orbital shaking. 30 μl of the 1+4 assay mixture previously prepared is added to each capture-antibody coated and blocked TopYield™ well. The samples are incubated overnight at 2-8° C. Four degrees is preferable, but if the temperature varies between 2-8 degrees, it has no detectable impact on the assay. Last, the samples are washed seven times with Wash-Buffer B (4×30 sec, 3×4 minutes) and twice for 1 minute with Wash-Buffer A.

### 2. Real-Time PCR

30 μl of PCR-Mastermix is pipetted into each well and the modules are sealed with Adhesive Foil. The sealed modules are transferred into a real-time PCR cycler. The PCR-program used is 5 minutes at 95° C. with one repeat; (30 seconds at 72° C., 12 seconds at 95° C., 30 seconds at 50° C.) with 45 repeats. The FAM (emission at 518 nm) is set as fluorophor.

### Controls:

A positive control is included in the PCR using 30 μl of the PCR mastermix spiked with 1 μl of the residual diluted Imperacer® conjugate and a negative control is included using only 30 μl PCR-Mastermix.

### 3. Calculation of Results

The real-time PCR-cycler records the increase of the normalized fluorescence signal (dR) for each cycle during DNA amplification. Subsequent to the run, an automatic baseline correction is applied by the software of the instrument. The instrument software is MXPRO Mx3005P v.3.20 from Stratagene 2006 in combination with Instrument OS v6.22 Control v4.40 Firmware v110.61 for instrument control and data analysis. In the next step, the software automatically calculates the threshold cycle (Ct), which represents the first PCR cycle at which the reporter signal exceeds the signal of a given uniform “Threshold”, manually set in the phase where signal increases linearly (typically 100-1000). A half-logarithmic plot of log dR against cycle number is used to choose the correct threshold value. To render an easy comparison of data obtained from real-time Imperacer™ and conventional ELISA, the problem has to be circumvented that Ct values are inversely proportional to antigen concentrations (0 antigen has the highest numerical value) while ELISA signals are directly proportional to antigen concentrations (0 antigen has the smallest numerical value). Therefore, □Ct values are calculated by subtracting the Ct values obtained for each signal from the total number of cycles carried out in the experiment.

4. Results from Immuo-PCR Assay

TABLE 1
Standard curves for each of the three sandwich assays.
The units of expression are in delta counts.

| pg/ml | | | |
|---|---|---|---|
| recombinant | | | |
| tau | Tau1/HT7 | DA9/HT7 | TG5/HT7 |
| 10000 | 31.66 | 31.91 | 34.68 |
| 2000 | 29.01 | 29.15 | 32.08 |
| 400 | 26.29 | 26.34 | 29.40 |
| 80 | 23.60 | 23.60 | 26.61 |
| 16 | 21.73 | 21.69 | 24.15 |
| 3.2 | 20.61 | 20.69 | 21.67 |
| 0.64 | 20.28 | 20.36 | 20.31 |
| 0 | 20.33 | 20.37 | 19.72 |

TABLE 2
Using point-point curve fitting from the standard curve, the amount of tau is determined
for each sample in each assay from the delta counts and is expressed in pg/ml.

| | | Tau1/HT7 | | DA9/HT7 | | TG5/HT7 | |
|---|---|---|---|---|---|---|---|
| Diagnosis | Serum Sample | pg/ml | Delta Count | pg/ml | Delta Count | pg/ml | Delta Count |
| AD | Sample B720 | 18.61 | 21.16 | * | 19.33 | 15.61 | 21.31 |
| AD | Sample B724 | 0.11 | 20.33 | 3.07 | 20.50 | 16.39 | 21.39 |
| AD | Sample B728 | 1.45 | 20.39 | * | 19.45 | 17.96 | 21.55 |
| AD | Sample B729 | 2.34 | 20.43 | * | 20.17 | 4.45 | 20.17 |
| AD | Sample B731 | * | 20.32 | 0.11 | 20.37 | 22.46 | 22.01 |
| AD | Sample B744 | * | 20.13 | * | 18.43 | 0.34 | 19.75 |
| AD | Sample B746 | * | 19.84 | * | 18.68 | 1.13 | 19.83 |
| AD | Sample B754 | * | 19.58 | * | 18.80 | * | 19.69 |
| AD | Sample B760 | 28.64 | 21.61 | * | 19.91 | 16.88 | 21.44 |
| AD | Sample B773 | * | 20.08 | * | 19.16 | 3.28 | 20.05 |
| AD | Sample B781 | * | 20.12 | * | 18.45 | 4.36 | 20.16 |
| AD | Sample B787 | * | 19.96 | * | 18.67 | * | 19.44 |
| AD | Sample B789 | 1.89 | 20.41 | * | 20.01 | 5.33 | 20.26 |
| AD | Sample B794 | * | 20.28 | 51.06 | 22.61 | 36.65 | 23.46 |
| AD | Sample B797 | 6.13 | 20.60 | * | 18.97 | * | 19.03 |
| AD | Sample B766 | 3.23 | 20.47 | 300.73 | 25.49 | 37.04 | 23.50 |
| AD | Sample B811 | * | 20.23 | 51.06 | 22.61 | 30.58 | 22.84 |
| AD | Sample B813 | 7.02 | 20.64 | * | 19.31 | 4.55 | 20.18 |
| Control | Sample B721 | 4.12 | 20.51 | * | 18.17 | * | 18.83 |
| Control | Sample B733 | 15.04 | 21.00 | 27.63 | 21.58 | 3.77 | 20.10 |
| Control | Sample B736 | 9.03 | 20.73 | 33.77 | 21.85 | 25.01 | 22.27 |
| Control | Sample B740 | 1.00 | 20.37 | * | 19.77 | * | 19.06 |
| Control | Sample B741 | * | 20.09 | 29.45 | 21.66 | 6.02 | 20.33 |
| Control | Sample B749 | * | 20.05 | 434.22 | 26.40 | 46.73 | 24.49 |
| Control | Sample B756 | 119.49 | 23.93 | 84.15 | 23.38 | 49.18 | 24.74 |
| Control | Sample B762 | 16.16 | 21.05 | 6.48 | 20.65 | 6.31 | 20.36 |
| Control | Sample B769 | 7328.31 | 30.77 | * | 33.20 | 1961.19 | 32.01 |
| Control | Sample B776 | 30.86 | 21.71 | * | 20.11 | 1.13 | 19.83 |
| Control | Sample B779 | 2.12 | 20.42 | * | 18.52 | * | 18.95 |
| Control | Sample B780 | 10.81 | 20.81 | * | 18.19 | * | 18.79 |
| Control | Sample B784 | * | 20.17 | * | 20.25 | * | 18.80 |
| Control | Sample B785 | * | 20.18 | * | 19.57 | * | 18.46 |

Asterisks (*) indicate that tau was not detectable.

TABLE 3
Ratio of DA9/Tau1 assays.

| | Serum | | | Ratio |
|---|---|---|---|---|
| Diagnosis | Sample | DA9 pg/ml | Tau 1 pg/ml | DA9/Tau1 |
| AD | B724 | 3.07 | 0.11 | 27.56 |
| AD | B766 | 300.73 | 3.23 | 93.07 |
| Control | B733 | 27.63 | 15.04 | 1.84 |
| Control | B736 | 33.77 | 9.03 | 3.74 |
| Control | B756 | 84.15 | 119.49 | 0.70 |
| Control | B762 | 6.48 | 16.16 | 0.40 |

TABLE 4
Ratio of TG5/Tau1 assays.

| | Serum | | | Ratio |
|---|---|---|---|---|
| Diagnosis | Sample | TG5 pg/ml | Tau 1 pg/ml | TG5/Tau1 |
| AD | B720 | 15.61 | 18.61 | 0.84 |
| AD | B724 | 16.39 | 0.11 | 147.16 |
| AD | B728 | 17.96 | 1.45 | 12.40 |
| AD | B729 | 4.45 | 2.34 | 1.90 |
| AD | B760 | 16.88 | 28.64 | 0.59 |
| AD | B789 | 5.33 | 1.89 | 2.82 |
| AD | B766 | 37.04 | 3.23 | 11.46 |
| AD | B813 | 4.55 | 7.02 | 0.65 |
| Control | B733 | 3.77 | 15.04 | 0.25 |
| Control | B736 | 25.01 | 9.03 | 2.77 |
| Control | B756 | 49.18 | 119.49 | 0.41 |
| Control | B762 | 6.31 | 16.16 | 0.39 |
| Control | B769 | 1961.19 | 7328.31 | 0.27 |
| Control | B776 | 1.13 | 30.86 | 0.04 |

TABLE 5
Ratio of TG5/DA9 assays.

| | Serum | | | Ratio |
|---|---|---|---|---|
| Diagnosis | Sample | TG5 pg/ml | DA9 pg/ml | TG5/DA9 |
| AD | B724 | 16.39 | 3.07 | 5.34 |
| AD | B731 | 22.46 | 0.11 | 197.49 |
| AD | B794 | 36.65 | 51.06 | 0.72 |
| AD | B766 | 37.04 | 300.73 | 0.12 |
| AD | B811 | 30.58 | 51.06 | 0.60 |
| Control | B733 | 3.77 | 27.63 | 0.14 |
| Control | B736 | 25.01 | 33.77 | 0.74 |
| Control | B741 | 6.02 | 29.45 | 0.20 |
| Control | B749 | 46.73 | 434.22 | 0.11 |
| Control | B756 | 49.18 | 84.15 | 0.58 |
| Control | B762 | 6.31 | 6.48 | 0.97 |

### 5. Analysis of Assay Data

For each sample and/or standard analysed in duplicate the mean values and standard deviation of □Ct are calculated. For quantification, □Ct of the calibration curve standards are plotted against the log and a linear regression is carried out.

Using the point-point curve fitting from the standard curve, the amount of tau is determined for each sample in each of the three assays and expressed in pg/ml. The raw data initially obtained from each assay is expressed in delta counts. The delta counts are converted to pg/ml using GraphPad Prism version 4.0c from GraphPad Software Inc, USA. Samples with no tau value or below the 0 point of the standard curve are excluded from further analysis. Following the calculation of the pg/ml per assay, ratios of the individual assays are constructed.

Receiver Operating Characteristic (ROC) curve analysis is performed for each of the single assays as well as using the ratio of two assays after the raw data is converted to pg/ml from the standard curves performed with each assay. Cutoff values were determined when the sum of specificity and sensitivity is maximized. ROC plots are generated using Analyse-it®2.20 from Analyse-it Software, Ltd., United Kingdom.

The data for each assay as well as the ratio of two assays are plotted in scattergrams. Cut-off values for detection of AD are determined by using the maximum of the sum of sensitivity and specificity identified in the ROC plot. The scattergrams are shown in FIGS. 2-7.

1. A method for determining the amount or presence of at least one tau protein or tau fragment in a biological sample comprising:
(a) contacting the sample with a first antibody and a second antibody to form a complex wherein both the first antibody and the second antibody bind specifically to tau polypeptide; and
(b) detecting the presence or amount of the complex, wherein the presence or amount of the complex is indicative of the presence or amount of tau polypeptides and wherein
(i) the first antibody is specific to the tau epitope 180-210 and the second antibody is specific to the tau epitope 150-170, or
(ii) the first antibody is specific to the tau epitope 192-204 and the second antibody is specific to the tau epitope 159-163, or
(iii) the first antibody is specific to the tau epitope 200-250 and the second antibody is specific to the tau epitope 150-170, or
(iv) the first antibody is specific to the tau epitope 210-230 and the second antibody is specific to the tau epitope 159-163, or
(v) the first antibody is specific to the tau epitope 102-150 and the second antibody is specific to the tau epitope 150-170, or
(vi) the first antibody is specific to the tau epitope 102-150 and the second antibody is specific to the tau epitope 159-163.

2. The method of claim 1, wherein the first antibody is specific to the tau epitope 180-210 and the second antibody is specific to the tau epitope 150-170.

3. The method of claim 1, wherein the first antibody is specific to the tau epitope 192-204 and the second antibody is specific to the tau epitope 159-163.

4. The method of claim 1, wherein the first antibody is specific to the tau epitope 200-250 and the second antibody is specific to the tau epitope 150-170.

5. The method of claim 1, wherein the first antibody is specific to the tau epitope 210-230 and the second antibody is specific to the tau epitope 159-163.

6. The method of claim 1, wherein the first antibody is specific to the tau epitope 102-150 and the second antibody is specific to the tau epitope 150-170.

7. The method of claim 1, wherein the first antibody is specific to the tau epitope 102-150 and the second antibody is specific to the tau epitope 159-163.

8. The method of claim 2, wherein the first antibody is specific to SEQ ID No. 1 and the second antibody is specific to SEQ ID No. 3.

9. The method of claim 3, wherein the first antibody is specific to SEQ ID No. 2 and the second antibody is specific to SEQ ID No. 4.

10. The method of claim 4, wherein the first antibody is specific to SEQ ID No. 5 and the second antibody is specific to SEQ ID No. 3.

11. The method of claim 5, wherein the first antibody is specific to SEQ ID No. 6 and the second antibody is specific to SEQ ID No. 4.

12. The method of claim 6, wherein the first antibody is specific to SEQ ID No. 7 and the second antibody is specific to SEQ ID No. 3.

13. The method of claim 7, wherein the first antibody is specific to SEQ ID No. 7 and the second antibody is specific to SEQ ID No. 4.

14. The method of claim 1, wherein the biological sample is whole blood, serum, plasma or cerebral spinal fluid.

15. (canceled)

16. The method of claim 1, further comprising the step of determining the ratio of at least two detected tau fragments or tau proteins to diagnosis a tauopathy or to rule out a tauopathy
wherein the ratio is one or more of
(i) the amount of tau detected when the first antibody is specific to the tau epitope 200-250 and the second antibody is specific to the tau epitope 150-170 relative to the amount of tau detected when the first antibody is specific to the tau epitope 180-210 and the second antibody is specific to the tau epitope 150-170,
(ii) the amount of tau detected when the first antibody is specific to the tau epitope 210-230 and the second antibody is specific to the tau epitope 159-163 relative to the amount of tau detected when the first antibody is specific to the tau epitope 192-204 and the second antibody is specific to the tau epitope 159-163,
(iii) the amount of tau detected when the first antibody is specific to the tau epitope 102-150 and the second antibody is specific to the tau epitope 150-170 relative to the amount of tau detected when the first antibody is specific to the tau epitope 180-210 and the second antibody is specific to the tau epitope 150-170,
(iv) the amount of tau detected when the first antibody is specific to the tau epitope 102-150 and the second antibody is specific to the tau epitope 159-163 relative to the amount of tau detected when the first antibody is specific to the tau epitope 192-204 and the second antibody is specific to the tau epitope 159-163,
(v) the amount of tau detected when the first antibody is specific to the tau epitope 200-250 and the second antibody is specific to the tau epitope 150-170 relative to the amount of tau detected when the first antibody is specific to the tau epitope 102-150 and the second antibody is specific to the tau epitope 150-170, and
(vi) the amount of tau detected when the first antibody is specific to the tau epitope 210-230 and the second antibody is specific to the tau epitope 159-163 relative to the amount of tau detected when the first antibody is specific to the tau epitope 102-150 and the second antibody is specific to the tau epitope 159-163.

17-22. (canceled)

23. The method of claim 16, wherein the tauopathy is Alzheimer's disease.

24. The method of claim 1, wherein detecting the amount of complex includes real-time PCR amplification of a polynucleic acid conjugated to the second antibody or enzyme amplification or readout by fluorescence or luminescence detection or colorimetric detection.

25-28. (canceled)

29. The methods of claim 1, wherein at least one of the first or second antibody is immobilized on a solid substrate and at least one of the first or second antibody comprises a label, wherein the presence or amount of the complex is measured by the amount of the signal from the label bound to the second antibody.

30. The method of claim 1, wherein at least one of the first antibody or the second antibody is a capture antibody and at least one of the first antibody or the second antibody is a detection antibody.

31-33. (canceled)

34. A method of detecting a tauopathy comprising
(a) determining the amount of a tau protein or tau fragment in a sample; and
(b) determining the ratio of at least two tau fragments or tau proteins.

35. A method of detecting tauopathy by determining the amount of a tau protein or a tau fragment in a biological sample comprising:
(a) contacting the sample with a first antibody and a second antibody to form a complex wherein both the first antibody and the second antibody bind specifically to tau polypeptide; and
(b) detecting the presence or amount of the complex wherein the presence or amount of the complex is indicative of the presence or amount of tau polypeptide; and
(c) determining the ratio of at least two detected tau fragments or tau proteins.

36. A method for determining the amount or presence of at least one tau protein or tau fragment in a biological sample comprising:
(a) contacting the sample with a first antibody and a second antibody to form a complex wherein both the first antibody and the second antibody bind specifically to tau polypeptide; and
(b) detecting the presence or amount of the complex, wherein the presence or amount of the complex is indicative of the presence or amount of tau polypeptide; and
(c) determining the ratio of at least two detected tau fragments or tau proteins to rule out Alzheimer's disease in a patient.


SEQUENCE LISTING (7 sequences)

SEQ ID NO: 1 - protein, 31 aa, Homo sapiens
KTPPSSGEPPKSGDRSGYSSPGSPGTPGSRS

SEQ ID NO: 2 - protein, 13 aa, Homo sapiens
GDRSGTSSPGSPG

SEQ ID NO: 3 - protein, 21 aa, Homo sapiens
KIATPRGAAPPGQKGQANATR

SEQ ID NO: 4 - protein, 5 aa, Homo sapiens
PPGQK

SEQ ID NO: 5 - protein, 51 aa, Homo sapiens
PGSPGTPGSRSRTPSLPTPPTREPKKVAVVRTPPKSPSSAKSRLQTAPVPM

SEQ ID NO: 6 - protein, 21 aa, Homo sapiens
SRTPSLPTPPTREPKKVAVVR

SEQ ID NO: 7 - protein, 49 aa, Homo sapiens
TAEEAGIGDTPSLEDEAAGHVTQARMVSKSKDGTGSDDKKAKGADGKTK